(19) United States
(12) Patent Application Publication
(10) Pub. No.: US 2021/0379354 A1
(43) Pub. Date: Dec. 9, 2021

(54) VENTRICULAR ASSISTANCE SYSTEM AND METHOD

(71) Applicant: Griffith University, Nathan (AU)

(72) Inventors: Geoff Tansley, Queensland (AU); Alice Catherine Boone, Queensland (AU)

(21) Appl. No.: 17/288,340

(22) PCT Filed: Oct. 30, 2019
(86) PCT No.: PCT/AU2019/051199

§ 371 Date: Apr. 23, 2021

(30) Foreign Application Priority Data
Oct. 31, 2018 (NL) 2021903

(51) Int. Cl. A61M 60/17; A61M 60/295; A61M 60/497; A61M 60/515; A61M 60/857; A61M 60/843

(52) U.S. Cl. CPC A61M 60/17; A61M 60/295; A61M 60/497; A61M 2230/04; A61M 60/857; A61M 60/843; A61M 2205/3331; A61M 60/515

(57) ABSTRACT

A system for providing ventricular assistance to a heart of a subject, the system including a balloon configured to be inserted into a ventricle of the heart, wherein the balloon is configured to differentially inflate to thereby urge blood towards a semilunar valve of the ventricle; a fluid conduit in fluid communication with the balloon; a pumping mechanism attached to the fluid conduit; and, a controller configured to control the pumping mechanism to thereby selectively supply fluid into the balloon so as to inflate the balloon at least partially in accordance with the cardiac cycle.

### BACKGROUND OF THE INVENTION

The present invention relates to a system and method for providing ventricular assistance to a heart of a subject, and in one example, to a system and method using a balloon that is inflated within a ventricle of the subject.

### DESCRIPTION OF THE PRIOR ART

The reference in this specification to any prior publication (or information derived from it), or to any matter which is known, is not, and should not be taken as an acknowledgment or admission or any form of suggestion that the prior publication (or information derived from it) or known matter forms part of the common general knowledge in the field of endeavour to which this specification relates.

Conditions such as heart failure result in a reduction in effectiveness of the heart, which can in turn lead to adverse health outcomes, including death. For example, dilated cardiomyopathy is characterised by Left Ventricular (LV) chamber enlargement and contractile dysfunction, whilst acute decompensated dilated cardiomyopathy, a Severe Heart Failure (SHF) condition, is characterised by a life-threatening decrease in cardiac output leading to a reduction in Ejection Fraction (EF)<40%.

In the event that pharmacological interventions are ineffective at managing such conditions, invasive alternatives, such as cardiac transplantation may be required. In many cases, due to comorbidities, time management issues and limited availability of organs for transplant, implantation of short-term mechanical circulatory support devices is required.

Such devices include blood pumps, such as rotary Ventricular Assist Devices (VADs). However, such devices are expensive, and difficult to install. Another example device is an intra-aortic balloon pump, which is one of the most extensively and routinely used short-term mechanical circulatory support devices. In this regard, the simplicity, minimal invasiveness and relatively low costs are the main factors encouraging the use and continuous improvement of intra-aortic balloon pumps. Intra-aortic balloon pumps are based on a volume displacement concept and consist of a balloon placed inside the aorta at the brachiocephalic root. Balloon inflation upon onset of cardiac diastole results in increased coronary perfusion and balloon deflation during systole results in decreased ventricular afterload. This short-term support can re-establish myocardial oxygen availability and consumption balance. Nonetheless, intra-aortic balloon pumps are not suitable in cases of low cardiac output; with limited ventricular unloading, they cannot independently support the systemic circulation.

A potential economical alternative to VADs and intra-aortic balloon pumps are Intra-Ventricular Balloon Pumps (IVBPs), which aim to support ventricular function by inflating a flexible chamber inside the ventricle.

U.S. Pat. No. 5,176,619 describes a heart-assist device which includes a flexible catheter carrying at least a ventricular balloon, such balloon corresponding in size and shape to the size and shape of the left ventricle in the heart being assisted, the ventricular balloon being progressively inflated creating a wave-like pushing effect and deflated synchronously and automatically by means of a control console which responds to heart signals from the catheter or elsewhere, the catheter optionally also carrying an aortic inflated and deflated automatically and synchronously (but in opposite phase) with the ventricular balloon by means of the control console to ensure high speed inflation-deflation.

However, existing attempts to utilise IVBPs have suffered complications, such as mitral valve regurgitation and atrial fibrillation, and as a result IVBPs are not widely used.

### SUMMARY OF THE PRESENT INVENTION

In one broad form, an aspect of the present invention seeks to provide a system for providing ventricular assistance to a heart of a subject, the system including: a balloon configured to be inserted into a ventricle of the heart, wherein the balloon is configured to differentially inflate to thereby urge blood towards a semilunar valve of the ventricle; a fluid conduit in fluid communication with the balloon; a pumping mechanism attached to the fluid conduit; and, a controller configured to control the pumping mechanism to thereby selectively supply fluid into the balloon so as to inflate the balloon at least partially in accordance with the cardiac cycle.

In one broad form, an aspect of the present invention seeks to provide a method for providing ventricular assistance to a heart of a subject, the method including: inserting a balloon into a ventricle of the heart, wherein the balloon is configured to differentially inflate to thereby urge blood towards a semilunar valve of the ventricle; providing a fluid conduit in fluid communication with the balloon; providing a pumping mechanism attached to the fluid conduit; and, using a controller to control the pumping mechanism to thereby selectively supply fluid into the balloon so as to inflate the balloon in accordance with the cardiac cycle.

In one broad form, an aspect of the present invention seeks to provide a method for providing ventricular assistance to a heart of a subject using a system including: a balloon configured to be inserted into a ventricle of the heart, wherein the balloon is configured to differentially inflate to thereby urge blood towards a semilunar valve of the ventricle; a fluid conduit in fluid communication with the balloon; a pumping mechanism attached to the fluid conduit; and, a controller, the method including using the controller to control the pumping mechanism to thereby selectively supply fluid into the balloon so as to inflate the balloon at least partially in accordance with the cardiac cycle.

In one broad form, an aspect of the present invention seeks to provide a computer program product for providing ventricular assistance to a heart of a subject using a system including: a balloon configured to be inserted into a ventricle of the heart, wherein the balloon is configured to differentially inflate to thereby urge blood towards a semilunar valve of the ventricle; a fluid conduit in fluid communication with the balloon; a pumping mechanism attached to the fluid conduit; and, a controller, wherein the computer program product includes computer executable code, which when executed by one or more suitably programmed electronic processing devices of the controller, causes the controller to control the pumping mechanism to thereby selectively supply fluid into the balloon so as to inflate the balloon at least partially in accordance with the cardiac cycle.

In one embodiment the balloon is configured to expand at least one of: longitudinally; and, towards the semilunar valve.

In one embodiment the balloon is configured to differentially inflate using at least one of: differential balloon wall thicknesses in different regions of the balloon; differential balloon wall materials in different regions of the balloon; balloon wall structures; ribbing; flow restrictions; internal walls; a mechanical restraint; an internal mesh; an external mesh; an external skin; and, separate inflatable portions.

In one embodiment the balloon includes a plurality of circumferential ribs spaced along a length of the balloon so that the balloon expands primarily longitudinally.

In one embodiment the balloon is configured to avoid interfering with operation of an atrioventricular valve of the ventricle.

In one embodiment the balloon is configured to avoid contact with at least one of: an atrioventricular valve complex; atrioventricular valve leaflets; atrioventricular valve papillary muscles; and, atrioventricular valve chordae.

In one embodiment when the balloon is inflated the balloon is shaped at least partially in accordance with a shape of the ventricle.

In one embodiment when the balloon is inflated the balloon includes at least one of: a length that is at least one of: dependent on a ventricular apex to atrioventricular valve distance; proportional to a ventricular apex to atrioventricular valve distance; approximately equal to a ventricular apex to atrioventricular valve distance; greater than 95% of a ventricular apex to atrioventricular valve distance; approximately 92% of a ventricular apex to atrioventricular valve distance; greater than 90% of a ventricular apex to atrioventricular valve distance; greater than 80% of a ventricular apex to atrioventricular valve distance; dependent on a ventricular apex to semilunar valve distance; approximately 20 mm less than a ventricular apex to semilunar valve distance; less than 100% of a ventricular apex to semilunar valve distance; less than 80% of a ventricular apex to semilunar valve distance; less than 75% of a ventricular apex to semilunar valve distance; less than 70% of a ventricular apex to semilunar valve distance; between 85 mm and 95 mm; between 80 mm and 100 mm; between 70 mm and 110 mm; between 40 mm and 100 mm; at least 40 mm; at least 60 mm; at least 70 mm; at least 80 mm; at least 85 mm; less than 120 mm; less than 110 mm; less than 100 mm; less than 95 mm; and, approximately 92 mm; a width that is at least one of: dependent on a ventricular apex to atrioventricular valve distance; proportional to a ventricular apex to atrioventricular valve distance; approximately half of the length; dependent on a ventricular apex to semilunar valve distance; approximately equal to 46% of the ventricular apex to atrioventricular valve distance; between 45% and 55% of the length; between 40% and 60% of the length; between 40 mm and 50 mm; between 35 mm and 55 mm; between 20 mm and 60 mm; at least 30 mm; at least 35 mm; at least 40 mm; less than 60 mm; less than 55 mm; less than 50 mm; and, approximately 44 mm; and, a depth that is at least one of: dependent on a ventricular apex to atrioventricular valve distance; approximately equal to 23% of the ventricular apex to atrioventricular valve distance; proportional to a ventricular apex to atrioventricular valve distance; approximately half of the width; approximately 25% of the length; between 45% and 55% of the width; between 40% and 60% of the width; between 20 mm and 25 mm; between 15 mm and 30 mm; at least 10 mm; at least 15 mm; at least 20 mm; less than 35 mm; less than 30 mm; less than 25 mm; and, approximately 23 mm.

In one embodiment when inflated the balloon has a volume of at least one of: dependent on a ventricular end-systolic volume; proportional to a ventricular end-systolic volume; approximately equal to a ventricular end-systolic volume; between 90% and 110% a ventricular end-systolic volume; between 80% and 120% a ventricular end-systolic volume; between 70% and 130% a ventricular end-systolic volume; at least 55 ml; at least 50 ml; at least 45 ml; less than 75 ml; less than 70 ml; less than 65 ml; and, approximately 60 ml.

In one embodiment the balloon includes an inlet bulb.

In one embodiment when inflated the inlet bulb has a radius of at least one of: proportional to a ventricular apex to semi-lunar valve distance; proportional to a ventricular apex to atrioventricular valve distance; radius a least 30% of a ventricular apex to atrioventricular valve distance; dependent on a ventricular apex to atrioventricular valve distance; proportional to a ventricular apex to atrioventricular valve distance; at least 30% of a ventricular apex to atrioventricular valve distance; at least 25% of a ventricular apex to atrioventricular valve distance; at least 20% of a ventricular apex to atrioventricular valve distance; greater than the depth of the balloon; less than the width of the balloon; at least 60% of the width of the balloon; at least 65% of the width of the balloon; less than 80% of the width of the balloon; less than 75% of the width of the balloon; approximately 70% of the width of the balloon; at least 130% of the depth of the balloon; at least 120% of the depth of the balloon; less than 150% of the depth of the balloon; less than 160% of the depth of the balloon; approximately 140% of the depth of the balloon; between 40% and 60% of the width; at least 20 mm; at least 25 mm; at least 30 mm; less than 45 mm; less than 40 mm; less than 35 mm; and, approximately 28 mm.

In one embodiment the inlet bulb expands at least one of: longitudinally; transversely; and, radially.

In one embodiment the balloon is configured to be inserted into the ventricle proximate a ventricular apex.

In one embodiment the balloon includes an inlet bulb configured to be positioned proximate to the ventricular apex.

In one embodiment the inlet bulb is configured to at least partially locate the balloon within the ventricle.

In one embodiment the balloon includes an inlet defining an inlet axis, and wherein in use the balloon extends in a direction that is at least one of: offset to the inlet axis; and, substantially parallel to but offset from the inlet axis.

In one embodiment the balloon is symmetric about an inlet axis to facilitate insertion of the balloon into the ventricle.

In one embodiment the controller monitors the cardiac cycle using signals from a sensor.

In one embodiment the controller uses signals from the sensor to determine at least one of: a phase of the cardiac cycle; onset of systole; onset of diastole; closure of a semi-lunar valve; and, closure of an atrioventricular valve. A system according to claim 17 or claim 18, wherein the sensor includes a heart activity sensor.

In one embodiment the sensor includes a flow sensor that senses at least one of: blood flow; and, a flow of fluid in the fluid conduit.

In one embodiment the sensor includes a pressure sensor that senses a pressure indicative of at least one of: a fluid pressure in a ventricle of the heart; a fluid pressure in the balloon; and, a fluid pressure in the fluid conduit.

In one embodiment the system includes a pressure sensor that senses a pressure of fluid within the balloon when the balloon is in an at least partially deflated state, and wherein the controller uses changes in the pressure to detect an onset of systole.

In one embodiment the controller controls the pumping mechanism to at least partially inflate the balloon at least one of: during systole; during transition; and, during diastole.

In one embodiment if the heart is in fibrillation, the controller controls the pumping mechanism to at least partially inflate the balloon independently of the cardiac cycle.

In one embodiment the controller controls the pumping mechanism so that the balloon reaches an end point of inflation at at least one of: at a defined phase of the cardiac cycle; at least 15% of the cardiac cycle from the onset of systole; at least 20% of the cardiac cycle from the onset of systole; approximately 25% of the cardiac cycle from the onset of systole; less than 30% of the cardiac cycle from the onset of systole; less than 35% of the cardiac cycle from the onset of systole; and, less than 40% of the cardiac cycle from the onset of systole.

In one embodiment the controller controls the pumping mechanism to inflate the balloon over a duty cycle that is at least one of: proportional to the duration of the cardiac cycle; at least 10% of the cardiac cycle; at least 15% of the cardiac cycle; approximately 20% of the cardiac cycle; less than 25% of the cardiac cycle; and, less than 30% of the cardiac cycle.

In one embodiment the controller controls the pumping mechanism to inflate the balloon over at least one of: a proportion of the cardiac cycle; at least 20% of the systolic phase; at least 30% of the systolic phase; at least 40% of the systolic phase; and, approximately 50% of the systolic phase.

In one embodiment the method includes identifying a duration of a current cardiac cycle based on at least one of: a length of a previous cardiac cycle; a length of at least two previous cardiac cycles; a first order derivative of a pressure signal; and, a first order derivative of a fluid flow signal.

In one embodiment the controller controls the pumping mechanism to adjust a total amount of inflation.

In one embodiment the controller is configured to control the pumping mechanism to at least partially deflate the balloon.

In one embodiment the balloon deflates at least partially passively.

In one embodiment the controller controls the pumping mechanism in accordance with at least one subject attribute.

In one embodiment the at least one subject attribute includes at least one of: a subject height; a subject weight; a medical symptom; a medical condition; and, a cardiac cycle status.

In one embodiment the controller: determines inflation parameters; and, controls inflation of the balloon in accordance with the inflation parameters.

In one embodiment the inflation parameters include at least one of: an inflation duration; an inflation amount; an inflation end point relative to the cardiac cycle; an inflation start point relative to the cardiac cycle; a deflation duration; a deflation amount; a deflation end point relative to the cardiac cycle; and, a deflation start point relative to the cardiac cycle.

In one embodiment the controller determines the inflation parameters using at least one of: signals from a sensor; at least one subject attribute; user input commands; and, stored inflation parameter profiles.

In one embodiment the controller includes: a memory that stores instructions; and, one or more electronic processing devices that operate in accordance with the instructions.

In one embodiment the memory stores at least one of: a balloon inflation history; events; and sensor readings.

In one embodiment the pumping mechanism includes at least one of: a fluid pump; a fluid reservoir; a positively pressurized fluid reservoir that is configured to inflate the balloon; and, a negatively pressurized fluid reservoir that is configured to deflate the balloon.

In one embodiment, the system includes: a pressure sensor configured to detect leaks in the balloon; and, a controller configured to control the balloon in accordance with signals from the sensor.

In one embodiment the balloon includes a double skin.

In one embodiment the method includes selecting one of a number of predetermined balloon configurations in accordance with at least one subject attribute.

It will be appreciated that the broad forms of the invention and their respective features can be used in conjunction and/or independently, and reference to separate broad forms is not intended to be limiting.

### BRIEF DESCRIPTION OF THE DRAWINGS

Various examples and embodiments of the present invention will now be described with reference to the accompanying drawings, in which: —

FIG. 1 is a schematic diagram of an example of a system for providing ventricular assistance to a heart of a subject;

FIG. 2 is a flow chart of an example of the operation of the system of FIG. 1;

FIG. 3A is a schematic side view of an example of a balloon for providing ventricular assistance to a heart of a subject in an inflated state;

FIG. 3B is a schematic front view of the balloon of FIG. 3A in the inflated state;

FIG. 3C is a schematic side view of the balloon of FIG. 3A in a partially deflated state;

FIG. 3D is a schematic front view of the balloon of FIG. 3A in the partially deflated state;

FIG. 3E is a schematic side view of an example of the balloon of FIG. 3A inflated within a ventricle;

FIG. 3F is a schematic side view of an alternative example of a balloon for providing ventricular assistance to a heart of a subject in an inflated state;

FIG. 3G is a schematic front view of the balloon of FIG. 3F in the inflated state;

FIG. 3H is a schematic side view of an further alternative example of a balloon for providing ventricular assistance to a heart of a subject in an inflated state;

FIG. 3I is a schematic side view of an further alternative example of a balloon for providing ventricular assistance to a heart of a subject in an inflated state;

FIG. 4 is a schematic diagram of a further example of a system for providing ventricular assistance to a heart of a subject;

FIGS. 5A and 5B are a flow chart of an example of the operation of the system of FIG. 4;

FIG. 6 is a schematic diagram of an example of a mock circulation loop;

FIG. 7A is a schematic diagram illustrating localisation of the main left ventricle landmarks;

FIG. 7B is a schematic diagram illustrating superimposition of normalised left ventricles;

FIG. 8A is a schematic side view of a specific example of a balloon for providing ventricular assistance to a heart of a subject in an inflated state and contained within a landing zone;

FIG. 8B is a schematic front view of the balloon of FIG. 8A in the inflated state and contained within a landing zone;

FIG. 8C is an image showing the balloon of FIG. 8A in a silicone ventricle of a mock circulation loop;

FIG. 9A is a graph illustrating an example of cardiac pressures measured in the mock circulation loop over a cardiac cycle for simulated Severe Heart Failure (SHF);

FIG. 9B is a graph illustrating an example of cardiac pressures measured in the mock circulation loop over a cardiac cycle for simulated Severe Heart Failure (SHF) with co-pulsation of an inflatable balloon;

FIG. 9C is a graph illustrating an example of cardiac pressures measured in the mock circulation loop over a cardiac cycle for simulated Severe Heart Failure (SHF) with transitional pulsation of an inflatable balloon;

FIG. 9D is a graph illustrating an example of cardiac pressures measured in the mock circulation loop over a cardiac cycle for simulated Severe Heart Failure (SHF) with counter-pulsation of an inflatable balloon;

FIG. 10A is a graph illustrating an example of aortic flow measured in the mock circulation loop for different balloon inflation conditions;

FIG. 10B is a graph illustrating an example of Mean Arterial Pressure (MAP) measured in the mock circulation loop for different balloon inflation conditions;

FIG. 10C is a graph illustrating an example of Left Ventricular End-Diastolic Volume (LVEDV) measured in the mock circulation loop for different balloon inflation conditions;

FIG. 10D is a graph illustrating an example of Ejection Fraction (EF) measured in the mock circulation loop for different balloon inflation conditions;

FIG. 11A is a graph illustrating an example of systolic period as a function of a balloon inflation end-point as a percentage of the cardiac cycle duration; and,

FIG. 11B is a graph illustrating an example of left ventricular peak pressure as a function of a balloon inflation end-point as a percentage of the cardiac cycle duration.

### DETAILED DESCRIPTION OF THE PREFERRED EMBODIMENTS

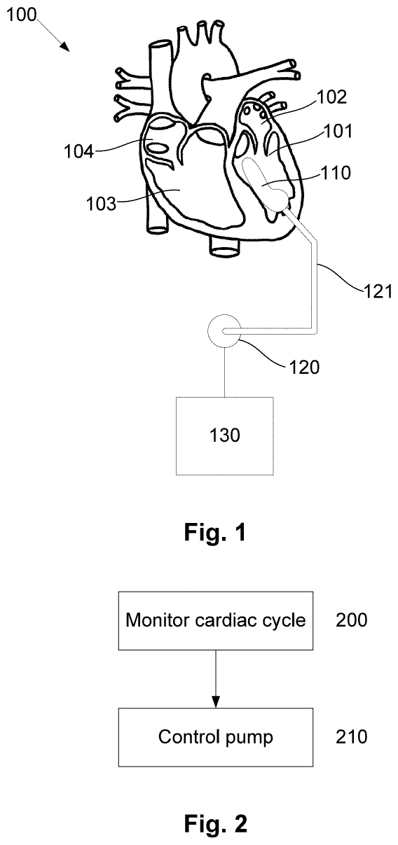

An example of a system for providing ventricular assistance to a heart of a subject will now be described with reference to FIG. 1.

For the purpose of illustration a heart 100 is shown including left and right ventricles 101, 103 and atriums 102, 103.

The system includes a balloon 110 configured to be inserted into a ventricle of the heart, with the ventricle selected depending on subject requirements. In this example, the balloon is inserted into the left ventricle 101 to provide pumping assistance to the systemic circulatory system, but the balloon could alternatively be inserted into the right ventricle 103 to provide pumping assistance to the pulmonary circulatory system, and similarly two balloons could be provided, with a respective balloon in each ventricle.

The balloon could be of any appropriate form, and could be any type of device that is able to inflate when filled with a fluid, including a gas and/or liquid, and it will therefore be appreciated that the term “balloon” is not intended to be limiting. The balloon could be made of any suitable material, but is typically made of a biocompatible flexible and optionally elastically expandable material. In one example, the balloon is made from silicone, although other suitable materials could be used.

Irrespective of the nature of the balloon 110, the balloon 110 is configured to differentially inflate to thereby urge blood towards a semilunar valve of the ventricle, and in particular the aortic valve in the case of the left ventricle, or the pulmonary valve in the case of the right ventricle.

The system further includes a fluid conduit 121, such as a catheter, or the like, in fluid communication with the balloon 110 and a pumping mechanism 120 attached to the fluid conduit, to allow a fluid to be pumped into the balloon 110, thereby causing the balloon to inflate. The pumping mechanism could be of any appropriate form, and could include a pump, such as an impeller or reciprocating pump, and/or could include pressurized reservoirs, as will be described in more detail below. In one example, the pumping mechanism 120 could be configured to operate reversibly, allowing fluid to be removed from the balloon, to thereby cause the balloon to deflate, although alternatively deflation could occur passively, as a result of pressure changes within the ventricle, or based on inherent resilience of the balloon. The fluid could include a liquid, but more typically is a gas as this provides compliance, allowing the balloon to expand or compress to accommodate pressure changes within the ventricle during the cardiac cycle. Whilst the gas could be air, more typically the gas is an inert gas, such as helium, carbon dioxide, or the like, is used to prevent biocompatibility issues in the event of fluid leakage through the balloon membrane. It will be appreciated from this that in one example, the pumping mechanism can be attached to or include a reservoir of gas, allowing gas to be supplied from and/or returned to the reservoir.

Whilst the pumping mechanism could be implanted, in other examples, the pumping mechanism and/or reservoir could be provided external to the subject, with the fluid conduit passing into the subject to deliver fluid to the balloon.

The system also typically includes a controller 130 that is configured to control the pumping mechanism. The nature of the controller 130 will vary depending on the preferred implementation, but typically the controller includes one or more electronic processing devices, such as microprocessors, microchip processors, logic gate configurations, firmware optionally associated with implementing logic such as an FPGA (Field Programmable Gate Array), or any other electronic device, system or arrangement.

An example of operation of the system will now be described with reference to FIG. 2.

In this example, at step 200, the controller 130 determines the cardiac cycle. This can be achieved by receiving signals from one or more sensors, such as a heart activity sensor, and/or flow or pressure sensors, as will be described in more detail below. At step 210, the controller 130 controls the pumping mechanism 120 to thereby selectively supply fluid into the balloon 110, so as to inflate the balloon 110 at least partially in accordance with the cardiac cycle. Specifically, in one preferred example, the balloon 110 is inflated during systole, to thereby assist operation of the ventricle by urging fluid towards the semilunar valve of the ventricle, and thereby expel blood from the ventricle. However, this is not essential and other modes of operation can be used, depending on the circumstances in which the system is being deployed.

In any event, it will be appreciated that in the above described arrangement describes a system that allows the heart to be assisted by selectively inflating a balloon within a ventricle to thereby urge blood from the ventricle via the respective semilunar valve. In one preferred example, the differential inflation can also be used to prevent interference of the balloon with papillary muscles, or valve chordae, thereby helping prevent disruption of an atrioventricular valve. Operation of the balloon is controlled in accordance with the cardiac cycle to thereby maximise the effectiveness of assistance provided. For example, this can be used to provide a pumping action during systole, or deflating the balloon during diastole can assist with ventricular filling.

A number of further features will now be described.

In one example the balloon is configured to expand towards the semilunar valve, thereby urging blood towards the valve. In a preferred example, this is achieved by having the balloon expand in a longitudinal direction, as this allows the balloon to sit within the ventricle, preferably proximate an apex of the ventricle, and then push blood through the ventricle towards the semilunar valve, as the balloon is inflated.

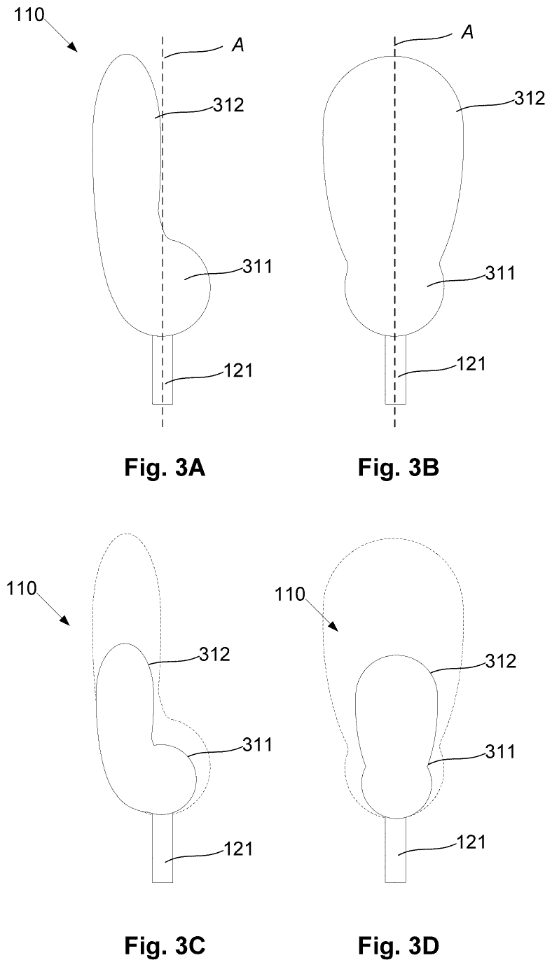

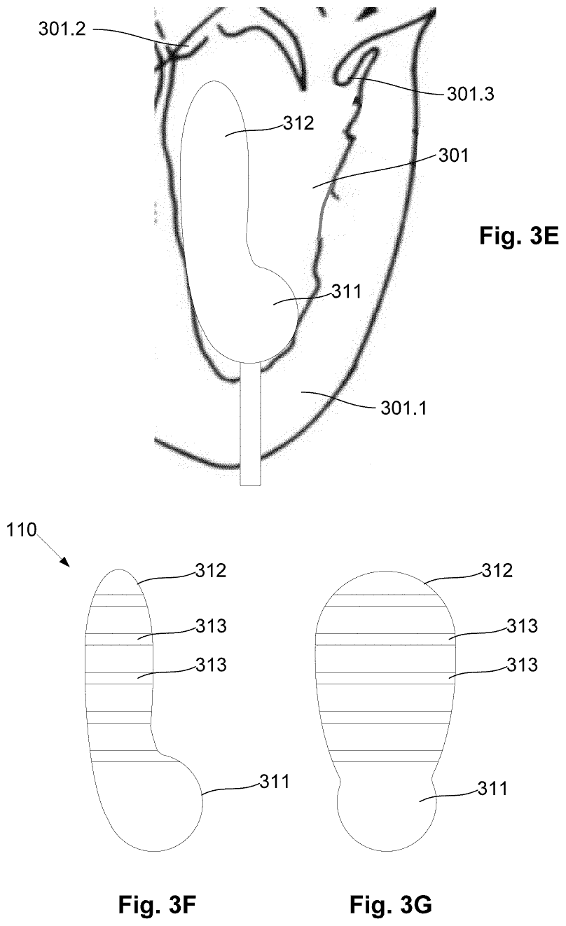

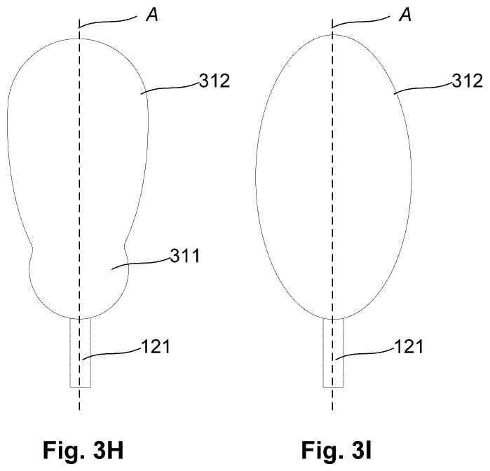

An example balloon configuration is shown in more detail in FIGS. 3A to 3E.

In this example, the balloon includes an inlet bulb 311, which is in fluid communication with the fluid conduit 121, and a main body 312, which extends from the inlet bulb 311. When inflated, as shown in FIGS. 3A and 3B, the body 312 is elongate and is generally flattened and relatively wide. This shape generally conforms to an internal shape of the ventricle, and allows the balloon to be positioned within the ventricle as shown in FIG. 3E. Specifically, in this example, the balloon is inserted into the ventricle 301 through the ventricular wall, so that the inlet bulb is positioned proximate to the ventricular apex 301.1. The shape of the inlet bulb 311 can be configured to assist locating the balloon within the ventricle so that the body 312 extends towards the semilunar valve 301.2.

During deflation, the balloon primarily contracts longitudinally, as shown in FIGS. 3C and 3D, so that during inflation, the balloon increases in length and pushes blood towards the semilunar valve 301.2, although it will be appreciated that radial contraction can additionally or alternatively be used. It should be noted that the representation shown in FIGS. 3C and 3D are of a partially deflated state, and it will be appreciated that in a fully deflated state the balloon can collapse down to a significantly smaller size, and in particular, will generally be a thin elongate body having a volume close to that of the balloon material. In contrast, the inlet bulb can expand longitudinally, transversely, and/or radially, to ensure the orientation of the balloon is maintained within the ventricle. This can also assist in driving fluid from the region surrounding the apex of the ventricle, which can in turn help reduce the likelihood of blood stagnation and clotting.

Differential expansion of the balloon can be achieved using a variety of different mechanisms, including using differential balloon wall thicknesses in different regions of the balloon, differential balloon wall materials in different regions of the balloon, balloon wall structures, ribbing, flow restrictions, internal walls, a mechanical restraint, an internal or external mesh, an external skin, separately inflatable portions, or the like.

For example, the balloon could include different regions each connected to the fluid conduit, which independently inflate, with the degree of inflation being controlled by a relative size of flow path into each region. In another example, a mechanical constraint, such as a mesh can be provided. Such a mesh could be embedded in the balloon wall, or provided externally to the balloon adapted to undergo limited expansion in one or more directions, in turn limiting expansion of the balloon, and thereby allowing the balloon to differentially inflate. The balloon may also include a dual skin, with the use of the second skin helping to prevent the balloon bursting or leaking. The second skin could also be adapted to provide mechanical constraint and thereby aid differential inflation of the balloon.

In one preferred example, shown in FIGS. 3F and 3G, the balloon includes a number of internal circumferential ribs 313, which can be formed from a thickening of the wall material, spaced along a length of the balloon so that the balloon expands primarily longitudinally, with lateral/radial expansion being limited by the ribs 313.

It will also be appreciated that alternative shapes could be used and that the shapes could be symmetric. For example, FIG. 3H shows an example of a balloon having a profile similar to that of the examples of FIG. 3A to 3G, albeit with the shape being symmetric about the inlet axis A, which can facilitate insertion of the balloon, specifically by avoiding the need for the balloon to be orientated in any particular direction. Alternatively, in the Examiner of FIG. 3I, the balloon is more ellipsoidal in shape. Similarly, a balloon could be provided that only includes a bulb portion. It will therefore be appreciated that a range of different shapes could be used and the examples of FIGS. 3A to 3G, whilst particularly effective, are not intended to be limiting.

The size and shape of the balloon is typically arranged to maintain a spacing from internal features of the ventricle, specifically avoiding contact with the atrioventricular valve complex, including the valve leaflets, papillary muscles, or valve chordae. This helps ensure correct operation of the valves, and in particular avoids obstruction of the atrioventricular valve, which in turn helps ensure correct ventricular function, and avoid mitral valve prolapse with concomitant regurgitation and insufficiency.

As part of this, a balloon inlet formed by the fluid conduit 121 defines an inlet axis A, with the balloon 110 extending in a direction that is offset to the inlet axis A, and in one particular example, substantially parallel to but offset from the inlet axis A, which assists with aligning the balloon body 312 with the semilunar valve, whilst spacing the inflated balloon from the papillary muscles and chordae.

Similarly, the size of the balloon could be configured to avoid the papillary muscles and chordae. In one example when the balloon is inflated the balloon includes a length that is dependent on a ventricular apex to atrioventricular valve distance and/or a ventricular apex to semilunar valve distance. In particular, the length can be proportional to a ventricular apex to atrioventricular valve distance, approximately equal to a ventricular apex to atrioventricular valve distance, greater than 95% of a ventricular apex to atrioventricular valve distance, approximately 92% of a ventricular apex to atrioventricular valve distance, greater than 90% of a ventricular apex to atrioventricular valve distance, greater than 80% of a ventricular apex to atrioventricular valve distance, dependent on a ventricular apex to semilunar valve distance, approximately 20 mm less than a ventricular apex to semilunar valve distance, less than 100% of a ventricular apex to semilunar valve distance, less than 80% of a ventricular apex to semilunar valve distance, less than 75% of a ventricular apex to semilunar valve distance, less than 70% of a ventricular apex to semilunar valve distance, between 85 mm and 95 mm, between 80 mm and 100 mm, between 70 mm and 110 mm, between 40 mm and 100 mm, at least 40 mm, at least 60 mm, at least 70 mm, at least 80 mm, at least 85 mm, less than 120 mm, less than 110 mm, less than 100 mm, less than 95 mm, or approximately 92 mm.

Similarly, the balloon typically has a width that is dependent on a ventricular apex to atrioventricular valve or semilunar valve distance. In one particular example, the width is proportional to a ventricular apex to atrioventricular valve distance, approximately half of the length, dependent on a ventricular apex to semilunar valve distance, approximately equal to 46% of the ventricular apex to atrioventricular valve distance, between 45% and 55% of the length, between 40% and 60% of the length, between 40 mm and 50 mm, between 35 mm and 55 mm, between 20 mm and 60 mm, at least 30 mm, at least 35 mm, at least 40 mm, less than 60 mm, less than 55 mm, less than 50 mm, or approximately 44 mm.

The balloon also typically has a depth that is dependent on a ventricular apex to atrioventricular or semilunar valve distance, and which is typically approximately equal to 23% of the ventricular apex to atrioventricular valve distance, proportional to a ventricular apex to atrioventricular valve distance, approximately half of the width, approximately 25% of the length, between 45% and 55% of the width, between 40% and 60% of the width, between 20 mm and 25 mm, between 15 mm and 30 mm, at least 10 mm, at least 15 mm, at least 20 mm, less than 35 mm, less than 30 mm, less than 25 mm, or approximately 23 mm. Alternatively, the width and depth might be identical, depending on the preferred implementation.

In a further example, when inflated the balloon has a volume that is dependent on a ventricular end-systolic volume, proportional to a ventricular end-systolic volume, approximately equal to a ventricular end-systolic volume, between 90% and 110% a ventricular end-systolic volume, between 80% and 120% a ventricular end-systolic volume, between 70% and 130% a ventricular end-systolic volume, at least 55 ml, at least 50 ml, at least 45 ml, less than 75 ml, less than 70 ml, less than 65 ml, or approximately 60 ml.

When inflated the inlet bulb has a radius that is proportional to a ventricular apex to semi-lunar valve distance, proportional to a ventricular apex to atrioventricular valve distance, dependent on a ventricular apex to atrioventricular valve distance, proportional to a ventricular apex to atrioventricular valve distance, at least 30% of a ventricular apex to atrioventricular valve distance, at least 25% of a ventricular apex to atrioventricular valve distance, at least 20% of a ventricular apex to atrioventricular valve distance, greater than the depth of the balloon, less than the width of the balloon, at least 60% of the width of the balloon, at least 65% of the width of the balloon, less than 80% of the width of the balloon, less than 75% of the width of the balloon, approximately 70% of the width of the balloon, at least 130% of the depth of the balloon, at least 120% of the depth of the balloon, less than 150% of the depth of the balloon, less than 160% of the depth of the balloon, approximately 140% of the depth of the balloon, between 40% and 60% of the width, at least 20 mm, at least 25 mm, at least 30 mm, less than 45 mm, less than 40 mm, less than 35 mm, or approximately 28 mm.

It will be appreciated from the above that the dimensions and shape of the balloon could be customized for individual subjects. In this example, a subject may undergo a scan, such as a computed tomography (CT) scan, allowing information regarding the shape of the ventricle to be derived, including the location of the atrioventricular valve and associated papillary muscles and chordae. The balloon could then be designed based on a template, scaling the balloon based on the dimensions and shape of the subject's ventricle, so that the size of the balloon is maximized for the available space, whilst ensuring contact with the papillary muscles, chordae or other internal features, is avoided.

In another example, a number of standard sizes of balloon could be produced, with the most appropriate balloon size being selected as needed. The selection could depend on one or more subject attributes, such as a subject height, a subject weight, a medical symptom, a medical condition, or the like. For example, medical conditions such as dilated cardiomyopathy, idiopathic, myocardial infarction, hypertrophy, or the like, can result in ventricles having different sizes and or shapes compared to that of a healthy heart. Accordingly, different balloons could be created for different medical conditions, with the balloons coming in different sizes, such as small, medium or large, for each condition. In this instance, balloon could be selected based on a medical condition and size of the subject. Whilst the balloon size may not be ideal for the subject, in this instance a degree of inflation can be used to tailor the balloon size making it suitable for the respective subject.

To achieve effective operation, in one example the controller determines inflation parameters, and, controls inflation of the balloon in accordance with the inflation parameters. The inflation parameters can include any one or more of an inflation duration (referred to generally as a duty cycle), an inflation amount or volume, an inflation start or end point relative to the cardiac cycle (referred to a phase), or the like. Although typically less important, deflation can also be controlled in a similar manner so that deflation is controlled depending on a deflation duration, a deflation amount, a deflation start or end point relative to the cardiac cycle, or the like. Alternatively, deflation can be performed passively, for example through displacement of the fluid from within the balloon during filling of the ventricle.

In one example the controller determines the inflation parameters using a variety of techniques, including using signals from a sensor, at least one subject attribute, user input commands, and, stored inflation parameter profiles. For example, typically the controller would have a number of stored inflation parameter profiles, with the profile being selected based on a medical condition, user input commands and/or signals from a sensor, to thereby optimize inflation for the current subject requirements. Parameters, such as an inflation volume can also be selected based on subject attributes, such as a subject size, in order to ensure the maximum balloon size during inflation is appropriate for the subject.

In one example the controller monitors the cardiac cycle using signals from a sensor, and uses this to determine parameters relating to the cardiac cycle and hence to control the pumping mechanism. Specifically, this can be used to determine a phase of the cardiac cycle, the onset of systole or diastole, closure or opening of a semi-lunar or atrioventricular valve, or the like.

The nature of the sensor will vary depending on the preferred implementation. For example, the sensor could include a heart activity sensor, such as an Electrocardiography (ECG) sensor, or similar. In another example, the sensor includes a flow sensor that senses either blood flow, or more typically a flow of fluid in the fluid conduit. Similarly, the sensor could include a pressure sensor that senses a pressure indicative of at least one of a fluid pressure in a ventricle of the heart, or more typically a fluid pressure in the balloon or fluid conduit.

Monitoring pressure or flow of fluid within the balloon or conduit can be used to detect the status of the cardiac cycle, for example as pressure within the fluid conduit will depend on pressures within the ventricle. So for example, an increase in fluid pressure in a partially deflated balloon could be indicative of ventricular filling during diastole. Thus, in one particular example, a pressure sensor is provided that senses fluid pressure within the balloon when the balloon is in a partially deflated state, with the controller then using changes in the fluid pressure to detect an onset of systole. Using a sensor in, or coupled to the fluid conduit, is particularly advantageous as this allows the sensor to be integrated into the system, avoiding the need for external sensors or similar, in order for the system to function correctly.

In one example the controller controls the pumping mechanism to at least partially inflate the balloon at least one of during systole (when the heart muscle contracts and pumps blood from the ventricle), during diastole (when the heart muscles relax allowing the ventricle to fill) and during transition (the time when the heart transitions from systole to diastole). For example, inflation during systole can assist expel blood from the ventricle and hence improve pumping effectiveness. However, in some circumstances inflation during transition or diastole can provide assistance in other manners. Similarly, if the heart is in fibrillation, the controller may need to control the pumping mechanism to at least partially inflate the balloon independently of the cardiac cycle, to thereby effectively replace functionality of the heart. In another example, the balloon could be configured to inflate on selected cardiac cycles, such as every other cycle, every third cycle, or the like, depending on the requirements.

In one example the controller controls the pumping mechanism so that the balloon reaches an end point of inflation at a defined phase of the cardiac cycle. Typically this is at least 15% of the cardiac cycle from the onset of systole, at least 20% of the cardiac cycle from the onset of systole, less than 30% of the cardiac cycle from the onset of systole, less than 35% of the cardiac cycle from the onset of systole, or less than 40% of the cardiac cycle from the onset of systole. Most typically, this is at approximately 25% of the cardiac cycle from the onset of systole, which can help maximize pumping effectiveness.

In one example the controller controls the pumping mechanism to inflate the balloon over a duty cycle that is proportional to the duration of the cardiac cycle. Typically this at least 10% of the cardiac cycle, at least 15% of the cardiac cycle, less than 25% of the cardiac cycle, or less than 30% of the cardiac cycle, and more typically is approximately 20% of the cardiac cycle.

In one example the controller controls the pumping mechanism to inflate the balloon over a proportion of the cardiac cycle and in particular at least 20% of the systolic phase, at least 30% of the systolic phase, at least 40% of the systolic phase, or approximately 50% of the systolic phase.

The controller can determine a duration of a current cardiac cycle using a variety of techniques, including but not limited to basing this on a length of a previous cardiac cycle, a length of at least two previous cardiac cycles, a first order derivative of a pressure signal, and, a first order derivative of a fluid flow signal.

The controller can also control the pumping mechanism to adjust a total amount of inflation as well as to control deflation of the balloon. Additionally and/or alternative deflation of the balloon could be performed passively, for example, allowing the balloon to deflate during diastole, as blood enters the ventricle and displaces fluid within the balloon.

As previously mentioned, in one example the controller controls the fluid pump in accordance with at least one subject attribute, such as a subject height, a subject weight, a medical symptom, a medical condition, or a cardiac cycle status, thereby allowing operation of the inflation process to be controlled to make this specific for the subject.

In one example, the controller includes a memory that stores instructions and one or more electronic processing devices that operate in accordance with the instructions, thereby allowing the system to be controlled using instructions forming part of software, firmware, or the like depending on the preferred implementation.

In one example, the controller can also be configured to store additional information in the memory, including but not limited to a balloon inflation history, including details of inflation times, durations and amounts, optionally recorded in conjunction with information regarding the heart activity, such as onset of systole, diastole, or the like, details of events, and/or sensor readings. This allows operation of the pump over time to be monitored, for example, to demonstrate the pump is functioning correctly and/or to allow the effectiveness of the assistance provided to be analysed and used to provide feedback and improve control.

As previously mentioned, the pumping mechanism could be of any appropriate form, and could include a fluid pump and/or fluid reservoir. In one example, a positively pressurized fluid reservoir can be configured to inflate the balloon, for example, allowing fluid under pressure to be supplied, with supply being controlled using a valve, such as a solenoid valve or similar, which in turn allows a set amount of fluid to be supplied rapidly. In this instance, the reservoir can be pressurised using a fluid pump, and as this occurs over a longer time period than the duration of inflation, this reduces pumping requirements associated with the pump, allowing smaller lighter pumps to be used, and reducing overall power usage, which is important with such wearable implanted system. Similarly, a negatively pressurized fluid reservoir can be used to deflate the balloon, with fluid being pumped between the reservoirs to re-pressurise the reservoirs after each balloon inflation/deflation cycle.

In one example, the system includes a pressure sensor configured to detect leaks in the balloon. For example, this can be used to detect a change in pressure within the balloon and compare this to an expected change of pressure can then fluid supplied to or removed from the balloon. In the event that a leak is detected, operation of the balloon could be halted thereby reducing the chance of fluid leaking into the subject. Additionally, the balloon can include a double skin, thereby reducing the likelihood of any fluid leaking into the subject.

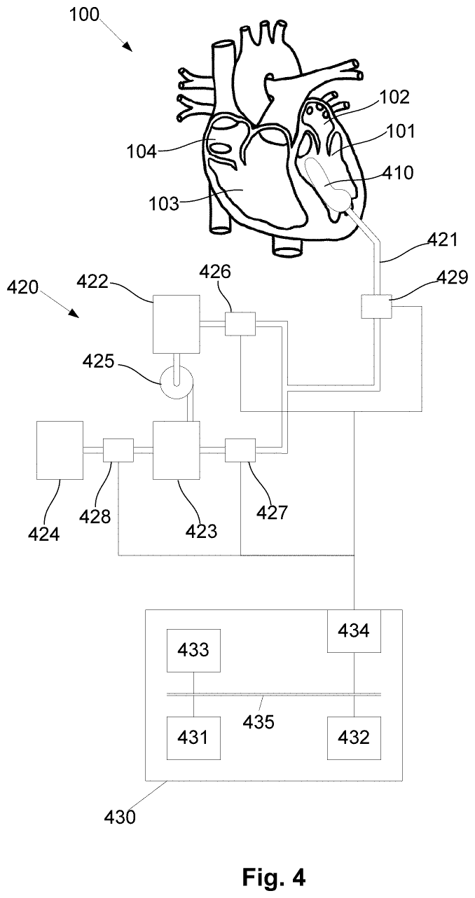

A specific example system will now be described in more detail with reference to FIG. 4.

In this example, the system includes a balloon 410 positioned in the left ventricle 101 of the subject's heart 100. The balloon 410 is connected via a catheter 421 to a pumping mechanism 420.

In this example, the pumping mechanism 420 includes reservoirs 422, 423, interconnected by a fluid pump 425, so that the reservoirs 422, 423 can be respectively negatively and positively pressurised in use. The reservoirs 422, 423 are connected to the catheter 421 via connecting pipes and associated control valves 426, 427, such that operation of the control vales 426, 427 can be used to allow negative pressure to be applied to the balloon 410 to assist deflation, or to allow pressurised fluid to be supplied to the balloon for inflation.

It will be appreciated that alternative approaches could however be used. For example, the pump could operate in forward and reverse directions, to alternate the pressurisation of the reservoirs, or a reciprocating pump could be used, to pressurise and subsequently depressurise the catheter 421 as needed.

A helium reservoir 424 is provided connected to the reservoir 423, via a connecting pipe and associated solenoid control valve 428, allowing the system to be re-filled with helium as needed, for example to replace helium lost through leakage through the balloon membrane, or the like. A pressure sensor 429 is provided, which senses a fluid pressure in the catheter 421.

The controller 430 includes at least one microprocessor 431, a memory 432, an optional input/output device 433, such as an optionally detachable keypad and/or touchscreen, and an external interface 434, interconnected via a bus 435 as shown. In this example the external interface 434 provides connectivity to the pump 423 and pressure sensor 425.

In use, the microprocessor 431 executes instructions in the form of applications software stored in the memory 432 to allow the required processes to be performed. The applications software may include one or more software modules, and may be executed in a suitable execution environment, such as an operating system environment, or the like. The memory also typically stores inflation parameters, optionally in the form of profiles, which can be selected based on user input commands, or based on signals received from the pressure sensor. The memory can also be used to store additional information, such as patient information and/or a history of operation and events, such as a pressure and heart rate history, allowing the supervising clinician to check system operation and/or perform a diagnostic assessment of current heart function.

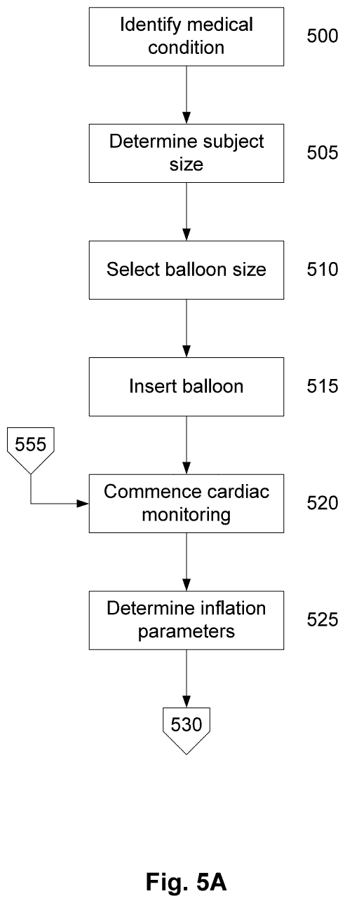

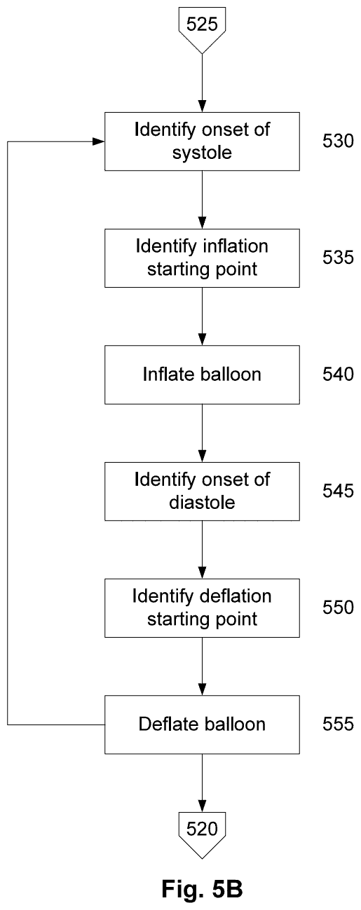

An example of the operation of the system will now be described with reference to FIGS. 5A and 5B. For the purpose of this example, it is assumed that the system is being used in an acute situation, where rapid treatment is necessary, and hence standard balloon configurations are used. However, it will be appreciated that similar approaches can be used in chronic situations, although this would also allow for the use of a customised balloon.

In this example, at step 500, the patient typically undergoes a review to identify a medical condition, which could be performed based on available symptoms and/or ECG or other similar measurements. At step 505, a subject size is determined, typically based on visual inspection, or similar. Using the medical condition and subject size, one of a number of different balloon configurations is selected, with the balloon being inserted into the ventricle at step 515. Such an insertion process can be performed by inserting a cannula into the ventricle and delivering the catheter and balloon through the cannula, although other suitable approaches could be used.

At step 520, monitoring of the cardiac cycle commences. This can be achieved by partially inflating the balloon and using pressure changes in the catheter in order to identify stages of the cardiac cycle, such as the onset of systole, diastole, or the like. Alternatively, this could be achieved by receiving data from a suitable sensor, such as an ECG or other sensor. Inflation parameters are determined at step 525, typically by retrieving a profile from memory, based on the identified medical condition and size of the subject, and optionally based on the results of the monitoring process, for example to take into account current heart activity.

Having established the cardiac cycle and inflation parameters, at step 530, the controller 430 monitors for the onset of systole, and then identifies an inflation point at which inflation should start at step 535, based on a defined phase and inflation duty cycle, relative to the onset of systole, as defined in the inflation parameters. At step 540, the balloon is inflated by opening the control valve 427, to allow positively pressurised helium to be supplied from the pressurised reservoir 423, thereby inflating the balloon. The control valve 427 is then closed once filling of the balloon is complete.

At step 545, the controller 430 monitors for the onset of diastole, and then identifies an deflation point at which deflation should start at step 550, based on a defined phase and deflation duty cycle, relative to the onset of diastole, as defined in the inflation parameters. At step 555, the balloon is deflated by opening the control valve 426, so that the helium is drawn into the negatively pressurised reservoir 422. Following this to the control valve 426 is closed, and the pump 425 actuated to pump helium from the reservoir 422, into the reservoir 423, and thereby restore pressures within the reservoirs 422, 423. At this point, overall pressure within the system can be monitored, with additional helium being supplied from the helium reservoir 424, by opening the control valve 428, if needed. It will be appreciated that this process effectively resets the pumping mechanism 420, allowing the process to return to step 530 to perform inflation and deflation for the next cardiac cycle.

In parallel, the system can periodically return to step 520, to allow the cardiac cycle to be monitored and inflation parameters adjusted if necessary. It will be appreciated that whilst this could be performed for each cardiac cycle, this is not necessarily required and may alternatively be performed periodically, such as every few minutes, hourly, or similar.

Accordingly, it will be appreciated that the above process allows the balloon to be selected from a range of standard balloons and then rapidly deployed and used to provide cardiac assistance making this suitable for use in acute circumstances. Nevertheless, the system can also be used in chronic situations, in order to provide long term support.

An example of a study to demonstrate the effectiveness of the above described system will now be described. In this example, the study aimed to develop a novel Intra-Ventricular Balloon Pump (IVBP) for short-term support of specific patient cohorts with SHF (e.g. dilated cardiomyopathy).

A silicone IVBP (balloon volume 60 mL), was designed and manufactured to avoid contact with internal Left Ventricular (LV) features (e.g. papillary muscles, aortic and mitral valves) based on LV computed tomography data of SHF patients with dilated cardiomyopathy (N=10). The haemodynamic effects of varying balloon inflation and deflation parameters (inflation duty (D) and phase from commencement of systole (ϕ) as percentages of the cardiac cycle) were evaluated in a custom-built systemic mock circulation loop. A SHF with dilated cardiomyopathy condition was simulated in the mock circulation loop, and the resulting IVBP effects on the haemodynamics were assessed.

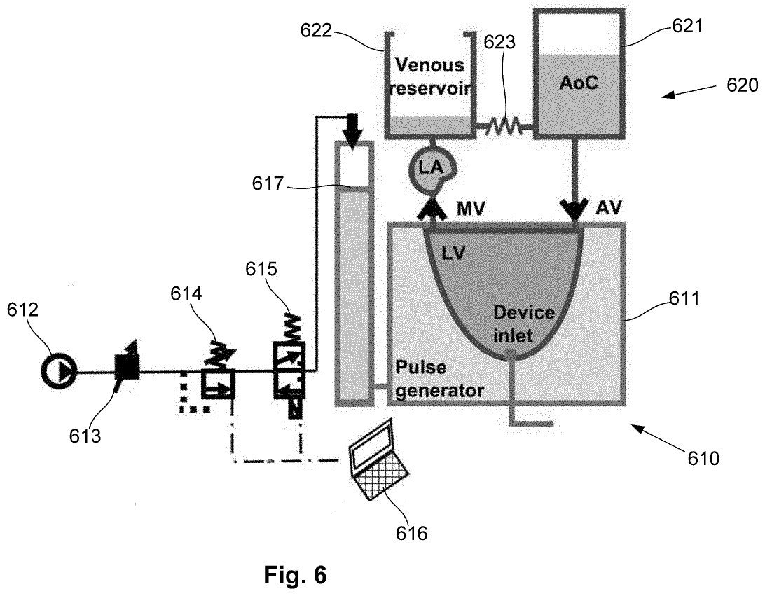

An example of a mock circulation loop to simulate systemic haemodynamics of SHF conditions is shown in FIG. 6.

In this example, the mock circulation loop includes two loops 610, 620. The first loop 610 includes a pulse generator, to create ventricular systole and allow passive ventricular filling, consisting of a chamber 611 filled with water and a pneumatic circuit, including a pressure source 612, a filter 613, a pressure regulator 614 and control valve 615, controlled by a computer 616 through Simulink (Matlab R2016a, MathWorks, Natick, US).

The second closed-loop 620 simulates the systemic circulation, based on a 3-element Windkessel model, including arterial compliance simulated by an air tight chamber 621 (height=260 mm, diameter=101.6 mm), and a preload reservoir chamber 622 (height=600 mm, diameter=101.6 mm) open to atmosphere, and a Systemic Vascular Resistance (SVR) 623 controlled by a pneumatic pinch valve (VMP 015.04K.71, AKO Ltd., Daventry, UK). The Systemic Vascular Resistance was calculated based on:

$$SVR = \frac{AoP - LAP}{AoF}$$

where:
AoP=Aortic Pressure [mmHg]
LAP=Left Atrial Pressure [mmHg] and
AoF=Aortic Flow [L/min].

Computed tomography images of a patient LV (end-diastolic volume=410 mL, Apex to Aortic valve centre axis (AA)=121 mm and Apex to Mitral valve centre axis (AM)=92 mm) and Left Atrium (LA), acquired at end-diastole, were reconstructed (Mimics v16.0, Materialise, Leuven, Belgium). The 3D LV and LA models were 3D printed (Acrylonitrile-Butadiene-Styrene, UP Plus2, Tiertime, Beijing, China) and post-processed to obtain a smooth surface rendering by sanding, acetone vapour smoothing and applying a coat of polyurethane (U-ECLEAR-VT, Barnes, Moorebank, Australia).

The flexible LV was moulded from silicone (Vario 40, Barnes, Moorebank, Australia) with 20% w/w diluent (AK100, Barnes, Moorebank, Australia) and the LA was moulded from silicone (Vario 15, Barnes, Moorebank, Australia) without diluent. During moulding, both the LV and LA were mounted onto a custom-made 2-axis rotational moulder (˜60 rev/min in both directions) to ensure uniform silicone distribution.

Umbrella silicone valves (diameter=35 mm) (UM 350.001 SD, Minivalves, Oldenzaal, The Netherlands) simulated the aortic and mitral valves. The blood analogue fluid used was a water/glycerol (60/40 by weight) mixture (3.5 mPa·s at 22° C.).

The system included an intraventricular balloon pump (IVBP) having a population-specific flexible balloon, an extracorporeal pneumatic pump and customised connecting elements.

Ten computed tomography images of patients with dilated LV scanned at end-diastole, were used for anatomical fitting analysis, with a single balloon geometry being designed based on this analysis. The anatomical fitting method was divided into four parts: (1) the recording of the LV measurements and landmarks, (2) the statistical analysis of these measurements, (3) the identification of the region of interest (i.e. landing zone) and (4) the fitting of a balloon to the landing zone—which is the desired volume within the ventricle which avoided the internal structures within the ventricle.

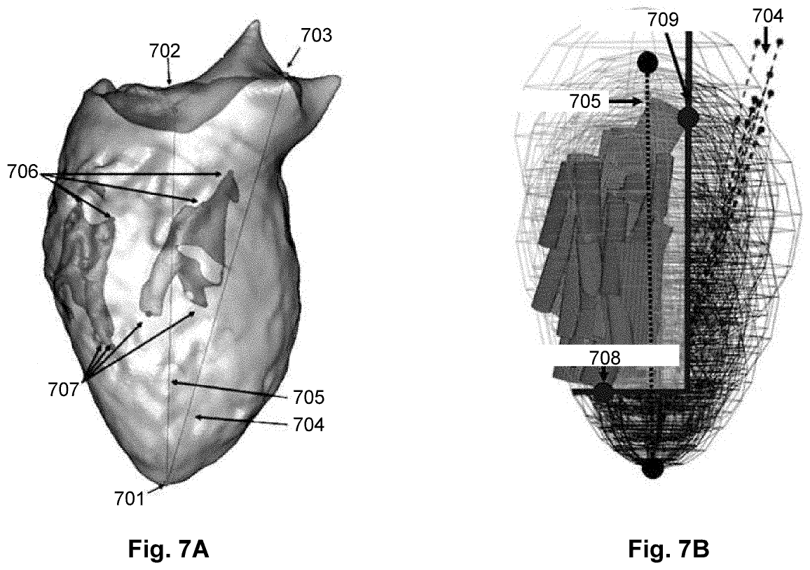

The main internal LV features are shown in FIG. 7A, including the LV apex 701, centre of the mitral and aortic valves 702, 703, main LV axes 704, 705 from the LV Apex to the Aortic centre (AA) and from the LV Apex to the Mitral centre (AM), tips and bases of the papillary muscles 706, 707. The diameter and coordinates of tip(s) 701 and base(s) 702 of the papillary muscles, and dimensions of the axes 704, 705, were measured. The landing zone of the balloon, i.e. the intraventricular region surrounding the subvalvular apparatus, was analytically defined by superimposing all normalised LV geometries and identifying the region extrema in FIG. 7B, thereby identify extrema points 708, 709.

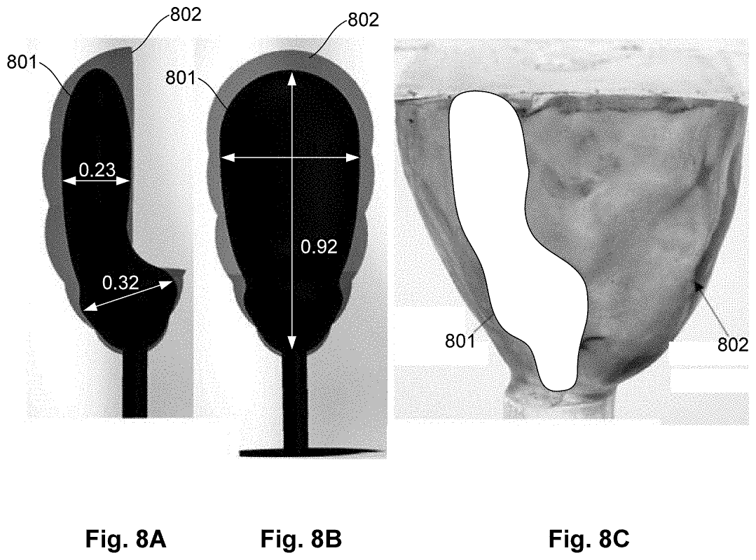

Ellipsoidal shapes were combined to fit within a calculated landing zone, which corresponds to a permissible region within which the balloon can operate without interfering with the function of the ventricle, resulting in the balloon geometry shown in FIGS. 8A and 8B. In this example, the balloon 801 is shown within the landing zone 800. The resultant balloon geometry was 3D printed (VeroWhite, Objet 24, Stratasys, Eden Prairie, US) and moulded from silicone (Vario 15, Barnes, Moorebank, Australia) in the rotational moulder (˜60 rev/min).

The balloon 801 was fixed inside the flexible patient-specific LV 802 as shown in FIG. 8C, and inflated with compressed air with the amplitude and timing controlled through an electropneumatic regulator 614 (ITV2030-012BS5, SMC Pneumatics, Tokyo, Japan) and a 3/2 way solenoid valve 615 (VT325-035DLS, SMC Pneumatics, Tokyo, Japan). The IVBP was operated by a custom-made Simulink program and synchronised with the mock circulation loop control architecture.

The mock circulation loop haemodynamics and IVBP pressures were recorded at 200 Hz (DS1104, dSpace, Paderborn, Germany). Four silicon-based transducers (PX181B-015C5V, Omega Engineering, Stamford, US) were used to measure LVP, AoP, LAP and the Balloon Pressure (BP). An in-line ultrasonic flow meter (TS410-13PXL, Transonic Systems, Ithaca, USA) was used to measure AoF. A magnetorestrictive level sensor (MTL-550 mm, Miran, Guangzhou, China) placed at the air/water interface 617 in the pulse generator was used to measure the LV volume variations.

The baseline SHF condition simulated the systemic haemodynamics clinical data of pre-LVAD implantation patients (HeartWare International, Inc., Framingham, US) (Muthiah et al. 2017). The SHF parameters corresponded to a depressed blood pressure, lowered AoF and EF and increased ventricular preload. The SVR was set to 1300 dyne·s·cm⁻⁵ (N·s·m⁻⁵) and the heart rate to 60 beats/min, which was a limitation of the mock circulation loop, but which would in practice be higher. The haemodynamic resulting from the IVBP support were compared to the post-LVAD implantation clinical data (Muthiah, K. et al., 2017. Longitudinal structural, functional, and cellular myocardial alterations with chronic centrifugal continuous-flow left ventricular assist device support. The Journal of heart and lung transplantation: the official publication of the International Society for Heart Transplantation, 36(7), pp. 722-731), as shown in Table 1 below.

TABLE 1

Conditions

| | Pre-LVAD | Post-LVAD |
|---|---|---|
| | implantation | implantation |
| Parameters | (mean ± std) | (mean ± std) |
| AoF [L/min] | 3.4 ± 1.4 | 5.0 ± 1.1 |
| MAP [mmHg] | 75.6 ± 11.4 | 80.3 ± 11.3 |
| LAP [mmHg] | 27.1 ± 6.6 | 14.8 ± 5.1 |
| EF [%] | 24 ± 8 | 35 ± 9 |

For determining the degree of support provided by the IVBP and the role of the balloon inflation timing on the SHF patient haemodynamics, 105 combinations of inflation duty cycles and phase delays were assessed. The inflation duty cycle (D), expressed as a percentage of the cardiac cycle duration, ranged from 10% to 30% by 5% increments. The inflation phase delay (ϕ), also expressed as a percentage of the cardiac cycle duration, was defined with respect to the start of ventricular systole (i.e. closing of the mitral valve) and ranged from 0% to 100% by 5% increments.

For each condition (D, ϕ), the main systemic haemodynamic parameters were recorded over 10 beats; for each time point of the cycle (200 points per cycle) the mean and standard deviation (N=10) were computed (Matlab r2017a, Mathworks, Natick, US). These haemodynamic parameters comprised Left Ventricular Pressure (LVP), Aortic Pressure (AoP), Left Atrial Pressure (LAP), Balloon Pressure (BP), Aortic Flow (AoF), LV End-Diastolic Volume (LVEDV), Stroke Volume (StV) and Ejection Fraction (EF). The LVEDV was defined as the maximum LV volume during each cycle and EF was defined as:

$$\frac{AoF}{LVEDV \cdot HR}$$

where: HR=heart rate.

The SHF haemodynamic baseline was measured over 10 cardiac cycles before each duty scenario (N=10 in five independent studies)—when the IVBP was turned off. Baseline values, mean and standard deviation at each time point (200 points per cardiac cycle) were computed. Mean AoF, MAP, EF and LVEDV computed over 10 cardiac cycles for each IVBP timing condition (D, ϕ) were compared to the SHF baseline.

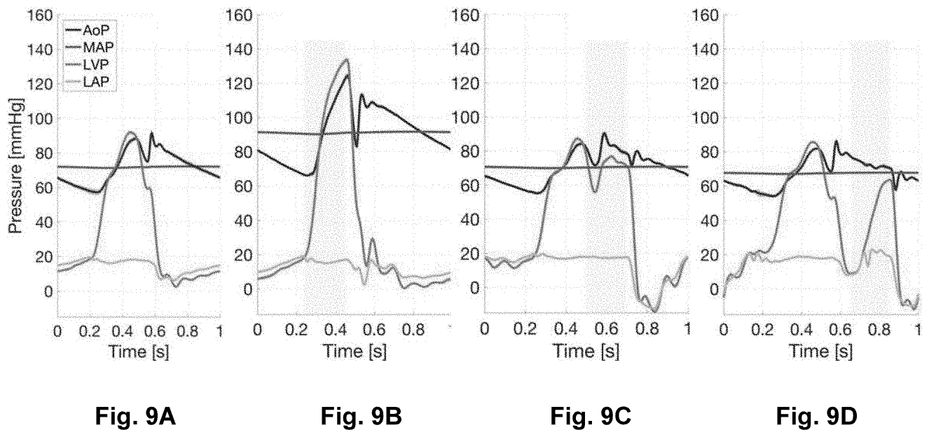

The mock circulation loop replicated the main haemodynamic features of a SHF patient as shown in FIGS. 9A to 9D. The simulated cycle presented the four main cardiac phases: isovolumetric contraction, ejection, isovolumetric relaxation and passive filling with systole spanning over 33% of the cycle. At closure of the aortic valve (around 0.55 s in FIG. 9Aa), AoP presented a hammering effect indicating aortic valve bouncing, also known as an aortic dicrotic notch.

When the SHF scenario was supported by the IVBP, the haemodynamic significantly varied with the pump timing (D, ϕ). Three types of balloon pulsation were identified: co-pulsation (FIG. 9B)—when the balloon was inflated in phase with respect to systole; counter-pulsation (FIG. 9D)—when the balloon was inflated out-of-phase with respect to systole and; transitional (FIG. 9C)—the phase between co- and counter-pulsation.

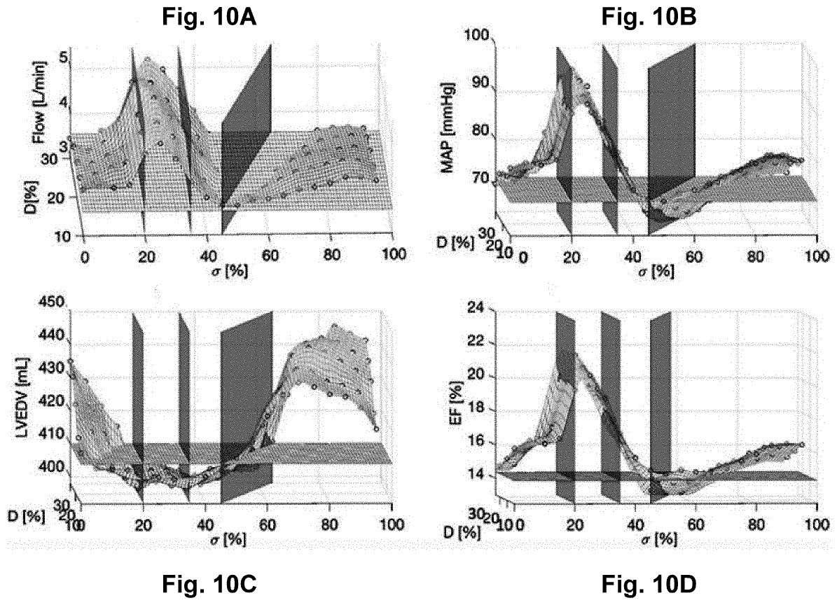

Pulsation types were defined by the balloon inflation end-point (σ=D+ϕ) and start-point (ϕ) with respect to start of systole in percentage of the cardiac cycle; co-pulsation corresponded to 20%≤σ≤35%, counter-pulsation corresponded to ϕ≥35% and σ<20% and the transition phase corresponded to σ>35% and ϕ<35%, with these pulsation types being outlined by the planes shown in FIGS. 10A to 1D, as described below.

Maximal and minimal values of the three pulsation types are presented in Table 2 and compared to the SHF baseline. Aortic Flow (AoF), Mean Arterial Pressure (MAP), Left Ventricular End-Diastolic Volume (LVEDV) and Ejection Fraction (EF).

TABLE 2

| | Conditions | | | |
|---|---|---|---|---|
| | | | | Counter- |
| | | | Transition | pulsation |
| | SHF | Co-pulsation | (σ > 35% and | (ϕ ≥ 35% and |
| Parameters | (base-line) | (20% ≤ σ ≤ 35%) | ϕ < 35%) | σ < 20%) |
| AoF | 3.5 | 4.2-5.2 | 3.1-4 | 3.1-4.3 |
| [L/min] | | (+20%-+49%) | (−11%-+14%) | (−11%-+23%) |
| MAP | 72 | 80-95 | 64-77 | 64-80 |
| [mmHg] | | (+11%-+32%) | (−11%-+7%) | (−11%-+11%) |
| LVEDV | 410 | 400-406 | 400-423 | 403-445 |
| [mL] | | −2%-−1%) | (−2%-+3%) | (−2%-+9%) |
| EF | 14.3 | 17.2-21.6 | 12.7-16.8 | 12.7-16.8 |
| [%] | | (20%-50%) | (−11%-+17%) | (−11%-+17%) |

FIGS. 10A to 10D depicts AoF, MAP, LVEDV and EF (averaged over 10 cycles) for all pump timing conditions as a function of the end-inflation point (σ), with planes shown in FIGS. 10A to 10D defining boundaries between the three timing conditions; co-pulsation (20%≤σ≤35%), counter-pulsation (ϕ≥35% and σ<20%) and the transition phase (σ>35% and ϕ<35%).

From this, it is apparent that in co-pulsation, the augmentation and decrease in ventricular pressure induced by the IVBP inflation and deflation, superposed to the existing LVP, impacted on the natural ventricular dynamics; in co-pulsation, aortic valve opening and closing timings varied with the IVBP actuation timing. Consequently, depending on the IVBP actuation timing, systolic duration increased or decreased when compared to the SHF baseline.

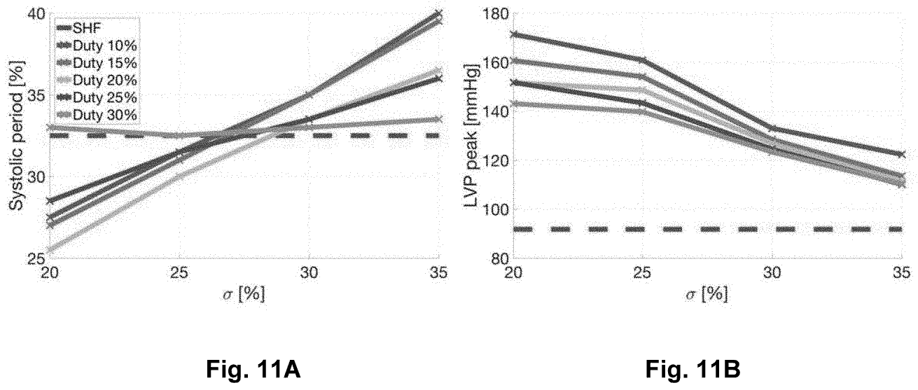

FIG. 11A presents systolic period as a function of the balloon inflation end-point (σ). For all co-pulsation conditions, except D=30%, the systolic period was positively correlated to σ: the sooner the balloon inflation ended, the shorter the systole was. The LVP peak was negatively correlated to the inflation end-point as shown in FIG. 11B.

Accordingly, the study demonstrates that depending on the actuation timing (D,ϕ), the system can result in improved haemodynamics and better supported the simulated failing ventricle. Two pump timings were identified, co-pulsation (20%≤σ≤35%) and counter-pulsation (ϕ≥35% and σ<20%) where σ=D+ϕ corresponds to the balloon end-inflation time.

IVBP co-pulsation resulted in increased Aortic Flow (AoF) from 3.5 L/min in SHF to up to 5.2 L/min, increased Mean Arterial Pressure (MAP) from 70 mmHg in SHF to up to 95 mmHg and increased Ejection Fraction (EF) from 14.3% by up to 21.6%. The balloon end-inflation time appeared to be the main determining factor in the degree of support; 6=25% optimised AoF, MAP and EF. Unfavourably, IVBP counter-pulsation resulted in a double pulse and increased Left-Ventricular End-Diastolic Volume (LVEDV) (by up to 9%), potentially impeding coronary perfusion, diastolic filling and myocardial recovery.

In counter-pulsation, the pressure increase in diastole generated by the balloon led to a second opening of the aortic valve (at 0.88 s in FIG. 9D). This counter-pulsation action impeded normal diastolic function: it disrupted LV filling and, it is expected that it will reduce coronary perfusion due to left coronary artery occlusion by the aortic valve leaflets and to the reduction in pressure gradient between the aorta and the myocardial tissues. Coronary perfusion, essential to cardiac recovery, is driven by the pressure gradient between the aorta and the myocardium (AoP and LVP), normally maximal in diastole. Hence, although AoF, MAP and EF of the SHF patient were improved by up to 20%, 13% and 15% respectively, during IVBP counter-pulsation, the LV loading, reflected by the increased LVEDV (9% larger when compared to baseline) and the presence of the double pulse during diastole provided evidence that counter pulsation would be less beneficial.

Increased filling pressure and/or increased afterload occurring in chronic heart failure result in myocardial dilation (cardiac remodelling); to support SHF patients, the IVBP must increase cardiac haemodynamics along with promoting reduction in LVEDV.

In co-pulsation, all haemodynamic parameters significantly improved compared to baseline: AoF (up to 5.2 L/min) and MAP (up to 95 mmHg) were comparable to the post-LVAD implantation patient values. Even though EF did not reach the targeted healthy EF value (>40%), the 3% decrease in LVEDV and 50% increase in EF, when compared to baseline, are promising results for cardiac function recovery. The patient LV model used in this study presented a large volume (end-diastolic volume=410 mL) when compared to average dilated cardiomyopathy LVs; the LVEDV and EF changes induced by the IVBP resulted in smaller ratios. Interestingly, despite the balloon duty cycle value (D), AoF, MAP, LVEDV and EF reached the approximately the same peaks (5.2 L/min, 95 mmHg, 400 mL and 21.6%, respectively) at the same end-inflation point corresponding to σ=25%. This observation indicated that the end-point of the balloon inflation was a key determinant in the level of haemodynamic support.

In co-pulsation, due to augmented ventricular pressure, systolic phase duration changed with the IVBP actuation timing; the systolic period became shorter when the balloon was inflated earlier. This systolic phase shortening induced lengthening of diastole providing more time for LV filling and coronary perfusion. IVBP co-pulsation with D=30% presented the least benefits in terms of systolic shortening indicating that this duty cycle was less favourable for cardiac support. Findings of previous IVBP studies where duty was D=33% and phase delay ϕ=0% could have therefore been improved by using different (D,ϕ) combinations; (D=20%, ϕ=5%) presenting the maximal haemodynamic support (AoF=5.2 L/min, MAP=95 mmHg, EF=21.6%) with 8% decrease in systolic period when compared to baseline and (D=20, ϕ=0%) presenting the shortest systolic period (22% systolic time reduction when compared to baseline) with improved haemodynamic support (AoF=4.8 L/min, MAP=90 mmHg and EF=20.2%).

It will be appreciated that above described results are specific to the simulated in-vitro condition, and that there would be differences when the system is implemented in a subject in practice. Thus the co-pulsation, counter-pulsation and transition boundary timing could be different in practice, and indeed may vary between different subjects and/or medical conditions, but that this could be easily ascertained through analysis similar to that described above.

Accordingly, the above described system can provide a cost-effective bridge-to-bridge solution to support decompensated heart failure patients, or could provide a bridge-to-recovery, bridge-to-candidacy, bridge-to decision, effectively supporting the ventricular function of the subject either until the subject recovers or an alternative long term solution can be found. The study demonstrated that a population-specific IVBP may be suitable to provide short-term mechanical circulatory support for SHF patients.

In one example, the device is specifically designed to fit the ventricular anatomy and avoid contacts with the subvalvular apparatus. In vitro analysis of the IVBP action on a simulated SHF patient proved that co-pulsation as opposed to counter-pulsation timing significantly improved the patient haemodynamic to values comparable to supported LVAD-patients. This in vitro study therefore proved the mechanical feasibility of the IVBP, potential limitations specific to cardiac physiology and architecture (e.g. mitral regurgitation) should be evaluated ex vivo or in vivo.

In one example, the IVB shape is designed to occupy as much end-systolic volume as possible for maximising systemic support while minimising outflow tract obstruction and interferences with the ventricular internal features.

The IVB shape may be designed to fit a specific patient cohort or an individual patient. In this regard, the IVB design is based on the cohort/individual's anatomical geometry. Anatomical fitting analysis can be performed using 3D ventricular models reconstructed from computed tomography images of end-systole ventricles of the patient cohort (e.g. dilated cardiomyopathy, acute myocardial infarction, etc.).

In one example, the IVB wall thickness or structural features can be controlled in such a way that the balloon inflates with a specific combination of motions (e.g. radial, longitudinal, torsion, etc.). The IVB can be designed to change shape proportionally to the inflation pressure.

The IVB inflation amplitude can be set based on the patient's BMI and/or BSA and on haemodynamic targets (e.g. cardiac output, arterial pressure, etc.), so that operation of the system is customised for the particular subject.

In one example, inflation and deflation timings can be defined by a duty cycle (D) and phase delay with commencement of ventricular systole (ϕ) as percentages of the cardiac cycle (wherein D+ϕ=25% in a preferred embodiment).

In one example, the system includes a means for monitoring ventricular and balloon pressures as well as gas flow at the inlet/outlet of the balloon catheter, which can assist with device control and operation.

The system can be used differently in different scenarios. For example, in acute situations time is a critical survival factor. Time between the sudden appearance of acute heart failure decompensation or myocardial ischemia and clinical treatment is known as the golden hour. Use of the system can provide a quick solution to salvage patients in acute failure—with the possibility of insertion by a paramedic to the patient to the hospital providing more time to clinicians to make a decision. Patients using this device will be ambulatory—meaning they will not take up valuable intensive care bed which will reduce the overall cost of their treatment. Such devices would not be bespoke. Typically, a set of balloon sizes could be provided, which would enable selection of the best fitting balloon ‘off the shelf’.

In chronic situations, patients on the organ waiting list are transplanted with a mechanical assist device normally used for permanent support. Due to the longevity, high-technology and implantation medical expertise, these devices are expensive (around $100,000). The short-term low-cost IVBP will decrease the total expenditure. Such devices could be bespoke, integrating patient-specific balloons that mirror the anatomy of a particular patient. Due to the chronic condition of the patient, high-resolution CT scans of a patient's heart are readily available, which enable the design of a bespoke balloon, utilising off the shelve design and 3D-printing software.

The term subject is intended to include animals, and more particularly humans, although this is not intended to be limiting and the techniques could be applied more broadly to other vertebrates and mammals.

Throughout this specification and claims which follow, unless the context requires otherwise, the word “comprise”, and variations such as “comprises” or “comprising”, will be understood to imply the inclusion of a stated integer or group of integers or steps but not the exclusion of any other integer or group of integers. As used herein and unless otherwise stated, the term “approximately” means ±20%.

Persons skilled in the art will appreciate that numerous variations and modifications will become apparent. All such variations and modifications which become apparent to persons skilled in the art, should be considered to fall within the spirit and scope that the invention broadly appearing before described.

The claims defining the invention are as follows:

1) A system for providing ventricular assistance to a heart of a subject, the system including:
a) a balloon configured to be inserted into a ventricle of the heart, wherein the balloon is configured to differentially inflate to thereby urge blood towards a semilunar valve of the ventricle;
b) a fluid conduit in fluid communication with the balloon;
c) a pumping mechanism attached to the fluid conduit; and,
d) a controller configured to control the pumping mechanism to thereby selectively supply fluid into the balloon so as to inflate the balloon at least partially in accordance with the cardiac cycle.

2) A system according to claim 1, wherein the balloon is configured to expand at least one of:
a) longitudinally; and,
b) towards the semilunar valve.

3) A system according to claim 1 or claim 2, wherein the balloon is configured to differentially inflate using at least one of:
a) differential balloon wall thicknesses in different regions of the balloon;
b) differential balloon wall materials in different regions of the balloon;
c) balloon wall structures;
d) ribbing;
e) flow restrictions;
f) internal walls;
g) a mechanical restraint;
h) an internal mesh;
i) an external mesh;
j) an external skin; and,
k) separate inflatable portions.

4) A system according to any one of the claims 1 to 3, wherein the balloon includes a plurality of circumferential ribs spaced along a length of the balloon so that the balloon expands primarily longitudinally.

5) A system according to any one of the claims 1 to 4, wherein the balloon is configured to avoid interfering with operation of an atrioventricular valve of the ventricle.

6) A system according to any one of the claims 1 to 5, wherein the balloon is configured to avoid contact with at least one of:
a) an atrioventricular valve complex;
b) atrioventricular valve leaflets;
c) atrioventricular valve papillary muscles; and,
d) atrioventricular valve chordae.

7) A system according to any one of the claims 1 to 6, wherein when the balloon is inflated the balloon is shaped at least partially in accordance with a shape of the ventricle.

8) A system according to any one of the claims 1 to 7, wherein when the balloon is inflated the balloon includes at least one of:
a) a length that is at least one of:
i) dependent on a ventricular apex to atrioventricular valve distance;
ii) proportional to a ventricular apex to atrioventricular valve distance;
iii) approximately equal to a ventricular apex to atrioventricular valve distance;
iv) greater than 95% of a ventricular apex to atrioventricular valve distance;
v) approximately 92% of a ventricular apex to atrioventricular valve distance;
vi) greater than 90% of a ventricular apex to atrioventricular valve distance;
vii) greater than 80% of a ventricular apex to atrioventricular valve distance;
viii) dependent on a ventricular apex to semilunar valve distance;
ix) approximately 20 mm less than a ventricular apex to semilunar valve distance;
x) less than 100% of a ventricular apex to semilunar valve distance;
xi) less than 80% of a ventricular apex to semilunar valve distance;
xii) less than 75% of a ventricular apex to semilunar valve distance;
xiii) less than 70% of a ventricular apex to semilunar valve distance;
xiv) between 85 mm and 95 mm;
xv) between 80 mm and 100 mm;
xvi) between 70 mm and 110 mm;
xvii) between 40 mm and 100 mm;
xviii) at least 40 mm;
xix) at least 60 mm;
xx) at least 70 mm;
xxi) at least 80 mm;
xxii) at least 85 mm;
xxiii) less than 120 mm;
xxiv) less than 110 mm;
xxv) less than 100 mm;
xxvi) less than 95 mm; and,
xxvii) approximately 92 mm;
b) a width that is at least one of:
i) dependent on a ventricular apex to atrioventricular valve distance;
ii) proportional to a ventricular apex to atrioventricular valve distance;
iii) approximately half of the length;
iv) dependent on a ventricular apex to semilunar valve distance;
v) approximately equal to 46% of the ventricular apex to atrioventricular valve distance;
vi) between 45% and 55% of the length;
vii) between 40% and 60% of the length;
viii) between 40 mm and 50 mm;
ix) between 35 mm and 55 mm;
x) between 20 mm and 60 mm;
xi) at least 30 mm;
xii) at least 35 mm;
xiii) at least 40 mm;
xiv) less than 60 mm;
xv) less than 55 mm;
xvi) less than 50 mm; and,
xvii) approximately 44 mm; and,
c) a depth that is at least one of:
i) dependent on a ventricular apex to atrioventricular valve distance;
ii) approximately equal to 23% of the ventricular apex to atrioventricular valve distance;
iii) proportional to a ventricular apex to atrioventricular valve distance;
iv) approximately half of the width;
v) approximately 25% of the length;
vi) between 45% and 55% of the width;
vii) between 40% and 60% of the width;
viii) between 20 mm and 25 mm;
ix) between 15 mm and 30 mm;
x) at least 10 mm;
xi) at least 15 mm;
xii) at least 20 mm;
xiii) less than 35 mm;
xiv) less than 30 mm;
xv) less than 25 mm; and,
xvi) approximately 23 mm.

9) A system according to any one of the claims 1 to 8, wherein when inflated the balloon has a volume of at least one of:
a) dependent on a ventricular end-systolic volume;
b) proportional to a ventricular end-systolic volume;
c) approximately equal to a ventricular end-systolic volume;
d) between 90% and 110% a ventricular end-systolic volume;
e) between 80% and 120% a ventricular end-systolic volume;
f) between 70% and 130% a ventricular end-systolic volume;
g) at least 55 ml;
h) at least 50 ml;
i) at least 45 ml;
j) less than 75 ml;
k) less than 70 ml;
l) less than 65 ml; and,
m) approximately 60 ml.

10) A system according to any one of the claims 1 to 9, wherein the balloon includes an inlet bulb.

11) A system according to claim 10, wherein when inflated the inlet bulb has a radius of at least one of:
a) proportional to a ventricular apex to semi-lunar valve distance;
b) proportional to a ventricular apex to atrioventricular valve distance;
c) radius a least 30% of a ventricular apex to atrioventricular valve distance;
d) dependent on a ventricular apex to atrioventricular valve distance;
e) proportional to a ventricular apex to atrioventricular valve distance;
f) at least 30% of a ventricular apex to atrioventricular valve distance;
g) at least 25% of a ventricular apex to atrioventricular valve distance;
h) at least 20% of a ventricular apex to atrioventricular valve distance;
i) greater than the depth of the balloon;
j) less than the width of the balloon;
k) at least 60% of the width of the balloon;
l) at least 65% of the width of the balloon;
m) less than 80% of the width of the balloon;
n) less than 75% of the width of the balloon;
o) approximately 70% of the width of the balloon;
p) at least 130% of the depth of the balloon;
q) at least 120% of the depth of the balloon;
r) less than 150% of the depth of the balloon;
s) less than 160% of the depth of the balloon;
t) approximately 140% of the depth of the balloon;
u) between 40% and 60% of the width;
v) at least 20 mm;
w) at least 25 mm;
x) at least 30 mm;
y) less than 45 mm;
z) less than 40 mm;
aa) less than 35 mm; and,
bb) approximately 28 mm.

12) A system according to claim 10 or claim 11, wherein the inlet bulb expands at least one of:
a) longitudinally;
b) transversely; and,
c) radially.

13) A system according to any one of the claims 1 to 12, wherein the balloon is configured to be inserted into the ventricle proximate a ventricular apex.

14) A system according to claim 13, wherein the balloon includes an inlet bulb configured to be positioned proximate to the ventricular apex.

15) A system according to claim 13 or claim 14, wherein the inlet bulb is configured to at least partially locate the balloon within the ventricle.

16) A system according to any one of the claims 1 to 15, wherein the balloon includes an inlet defining an inlet axis, and wherein in use the balloon extends in a direction that is at least one of:
a) offset to the inlet axis; and,
b) substantially parallel to but offset from the inlet axis.

17) A system according to any one of the claims 1 to 16, wherein the balloon is symmetric about an inlet axis to facilitate insertion of the balloon into the ventricle.

18) A system according to any one of the claims 1 to 17, wherein the controller monitors the cardiac cycle using signals from a sensor.

19) A system according to claim 18, wherein the controller uses signals from the sensor to determine at least one of:
a) a phase of the cardiac cycle;
b) onset of systole;
c) onset of diastole;
d) closure of a semi-lunar valve; and,
e) closure of an atrioventricular valve.

20) A system according to claim 18 or claim 19, wherein the sensor includes a heart activity sensor.

21) A system according to any one of the claims 1 to 20, wherein the sensor includes a flow sensor that senses at least one of:
a) blood flow; and,
b) a flow of fluid in the fluid conduit.

22) A system according to any one of the claims 17 to 21, wherein the sensor includes a pressure sensor that senses a pressure indicative of at least one of:
a) a fluid pressure in a ventricle of the heart;
b) a fluid pressure in the balloon; and,
c) a fluid pressure in the fluid conduit.

23) A system according to any one of the claims 1 to 22, wherein the system includes a pressure sensor that senses a pressure of fluid within the balloon when the balloon is in an at least partially deflated state, and wherein the controller uses changes in the pressure to detect an onset of systole.

24) A system according to any one of the claims 1 to 23, wherein the controller controls the pumping mechanism to at least partially inflate the balloon at least one of:
a) during systole;
b) during transition; and,
c) during diastole.

25) A system according to any one of the claims 1 to 24, wherein if the heart is in fibrillation, the controller controls the pumping mechanism to at least partially inflate the balloon independently of the cardiac cycle.

26) A system according to any one of the claims 1 to 25, wherein the controller controls the pumping mechanism so that the balloon reaches an end point of inflation at at least one of:
a) at a defined phase of the cardiac cycle;
b) at least 15% of the cardiac cycle from the onset of systole;
c) at least 20% of the cardiac cycle from the onset of systole;
d) approximately 25% of the cardiac cycle from the onset of systole;
e) less than 30% of the cardiac cycle from the onset of systole;
f) less than 35% of the cardiac cycle from the onset of systole; and,
g) less than 40% of the cardiac cycle from the onset of systole.

27) A system according to any one of the claims 1 to 26, wherein the controller controls the pumping mechanism to inflate the balloon over a duty cycle that is at least one of:
a) proportional to the duration of the cardiac cycle;
b) at least 10% of the cardiac cycle;
c) at least 15% of the cardiac cycle;
d) approximately 20% of the cardiac cycle;
e) less than 25% of the cardiac cycle; and,
f) less than 30% of the cardiac cycle.

28) A system according to any one of the claims 1 to 27, wherein the controller controls the pumping mechanism to inflate the balloon over at least one of:
a) a proportion of the cardiac cycle;
b) at least 20% of the systolic phase;
c) at least 30% of the systolic phase;
d) at least 40% of the systolic phase; and,
e) approximately 50% of the systolic phase.

29) A system according to any one of the claims 1 to 28, wherein the method includes identifying a duration of a current cardiac cycle based on at least one of:
a) a length of a previous cardiac cycle;
b) a length of at least two previous cardiac cycles;
c) a first order derivative of a pressure signal; and,
d) a first order derivative of a fluid flow signal.

30) A system according to any one of the claims 1 to 29, wherein the controller controls the pumping mechanism to adjust a total amount of inflation.

31) A system according to any one of the claims 1 to 30, wherein the controller is configured to control the pumping mechanism to at least partially deflate the balloon.

32) A system according to any one of the claims 1 to 31, wherein the balloon deflates at least partially passively.

33) A system according to any one of the claims 1 to 32, wherein the controller controls the pumping mechanism in accordance with at least one subject attribute.

34) A system according to claim 33, wherein the at least one subject attribute includes at least one of:
a) a subject height;
b) a subject weight;
c) a medical symptom;
d) a medical condition; and,
e) a cardiac cycle status.

35) A system according to any one of the claims 1 to 34, wherein the controller:
a) determines inflation parameters; and,
b) controls inflation of the balloon in accordance with the inflation parameters.

36) A system according to claim 35, wherein the inflation parameters include at least one of:
a) an inflation duration;
b) an inflation amount;
c) an inflation end point relative to the cardiac cycle;
d) an inflation start point relative to the cardiac cycle;
e) a deflation duration;
f) a deflation amount;
g) a deflation end point relative to the cardiac cycle; and,
h) a deflation start point relative to the cardiac cycle.

37) A system according to claim 35 or claim 36, wherein the controller determines the inflation parameters using at least one of:
a) signals from a sensor;
b) at least one subject attribute;
c) user input commands; and,
d) stored inflation parameter profiles.

38) A system according to any one of the claims 1 to 37, wherein the controller includes:
a) a memory that stores instructions; and,
b) one or more electronic processing devices that operate in accordance with the instructions.

39) A system according to claim 38, wherein the memory stores at least one of:
a) a balloon inflation history;
b) events; and
c) sensor readings.

40) A system according to any one of the claims 1 to 39, wherein the pumping mechanism includes at least one of:
a) a fluid pump;
b) a fluid reservoir;
c) a positively pressurized fluid reservoir that is configured to inflate the balloon; and,
d) a negatively pressurized fluid reservoir that is configured to deflate the balloon.

41) A system according to any one of the claims 1 to 40, wherein the system includes:
a) a pressure sensor configured to detect leaks in the balloon; and,
b) a controller configured to control the balloon in accordance with signals from the sensor.

42) A system according to any one of the claims 1 to 41, wherein the balloon includes a double skin.

43) A method for providing ventricular assistance to a heart of a subject, the method including:
a) inserting a balloon into a ventricle of the heart, wherein the balloon is configured to differentially inflate to thereby urge blood towards a semilunar valve of the ventricle;
b) providing a fluid conduit in fluid communication with the balloon;
c) providing a pumping mechanism attached to the fluid conduit; and,
d) using a controller to control the pumping mechanism to thereby selectively supply fluid into the balloon so as to inflate the balloon in accordance with the cardiac cycle.

44) A method according to claim 43, wherein the method includes selecting one of a number of predetermined balloon configurations in accordance with at least one subject attribute.

45) A method according to claim 43 or claim 44, wherein the method includes controlling the pumping mechanism to adjust a total amount of inflation in accordance with at least one subject attribute.

46) A method according to claim 44 or claim 45, wherein the at least one subject attribute includes at least one of:
a) a subject height;
b) a subject weight;
c) a medical symptom;
d) a medical condition; and,
e) a cardiac cycle status.

47) A method according to any one of the claims 43 to 46, wherein the method is performed using the system of any one of the claims 1 to 42.

48) A method for providing ventricular assistance to a heart of a subject using a system including:
a) a balloon configured to be inserted into a ventricle of the heart, wherein the balloon is configured to differentially inflate to thereby urge blood towards a semilunar valve of the ventricle;
b) a fluid conduit in fluid communication with the balloon;
c) a pumping mechanism attached to the fluid conduit; and,
d) a controller, the method including using the controller to control the pumping mechanism to thereby selectively supply fluid into the balloon so as to inflate the balloon at least partially in accordance with the cardiac cycle.

49) A method according to claim 48, wherein the controller:
a) determines inflation parameters; and,
b) controls inflation of the balloon in accordance with the inflation parameters.

50) A system according to claim 49, wherein the inflation parameters include at least one of:
a) an inflation duration;
b) an inflation amount;
c) an inflation end point relative to the cardiac cycle;
d) an inflation start point relative to the cardiac cycle;
e) a deflation duration;
f) a deflation amount;
g) a deflation end point relative to the cardiac cycle; and,
h) a deflation start point relative to the cardiac cycle.

51) A system according to claim 49 or claim 50, wherein the controller determines the inflation parameters using at least one of:
a) signals from a sensor;
b) at least one subject attribute;
c) user input commands; and,
d) stored inflation parameter profiles.

52) A method according to any one of the claims 48 to 51, wherein the method is performed using the system of any one of the claims 1 to 42.

53) A computer program product for providing ventricular assistance to a heart of a subject using a system including:
a) a balloon configured to be inserted into a ventricle of the heart, wherein the balloon is configured to differentially inflate to thereby urge blood towards a semilunar valve of the ventricle;
b) a fluid conduit in fluid communication with the balloon;
c) a pumping mechanism attached to the fluid conduit; and,
d) a controller, wherein the computer program product includes computer executable code, which when executed by one or more suitably programmed electronic processing devices of the controller, causes the controller to control the pumping mechanism to thereby selectively supply fluid into the balloon so as to inflate the balloon at least partially in accordance with the cardiac cycle.

54) A computer program product according to claim 53, wherein the computer program product causes the controller to perform the method of any one of the claims 48 to 52.
